SEQUENCE LISTING (2 sequences)

SEQ ID NO: 1 - protein, 240 aa, Homo sapiens
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 2 - DNA, 2894 bp, Homo sapiens
cgagactccatctcaaaaacaaaacaaataaacgaacaaaaaaacccacaacgtattattttcttgtttacgaggtttcttgtctctctggctccaccagaagaggagcagggacccttcttgctgttgttcattgctgcatcccccacaccgagagcagagcctggcatgggcagaaagtcctcagtcgatatttggtggccccaagcgaatgaagcatccaagaagggaaagctgggggctccccactgcacttgccacctgagtcacattttcagaagcctctggaaagtcgtgcacagcccaggagtgttgagcaatttcggtttcctctgaggttgaaggacccaggcgtgtcagccctgctccagacaccttgggcatggaggagagtgtcgtacggccctcagtgtttgtggtggatggacagaccgacatcccattcacgaggctgggacgaagccaccggagacagtcgtgcagtgtggcccgggtgggtctgggtctcttgctgttgctgatgggggccgggctggccgtccaaggctggttcctcctgcagctgcactggcgtctaggagagatggtcacccgcctgcctgacggacctgcaggctcctgggagcagctgatacaagagcgaaggtctcacgaggtcaacccagcagcgcatctcacaggggccaactccagcttgaccggcagcggggggccgctgttatgggagactcagctgggcctggccttcctgaggggcctcagctaccacgatggggcccttgtggtcaccaaagctggctactactacatctactccaaggtgcagctgggcggtgtgggctgcccgctgggcctggccagcaccatcacccacggcctctacaagcgcacaccccgctaccccgaggagctggagctgttggtcagccagcagtcaccctgcggacgggccaccagcagctcccgggtctggtgggacagcagcttcctgggtggtgtggtacacctggaggctggggagaaggtggtcgtccgtgtgctggatgaacgcctggttcgactgcgtgatggtacccggtcttacttcggggctttcatggtgtgaaggaaggagcgtggtgcattggacatgggtctgacacgtggagaactcagagggtgcctcaggggaaagaaaactcacgaagcagaggctgggcgtggtggctctcgcctgtaatcccagcactttgggaggccaaggcaggcggatcacctgaggtcaggagttcgagaccagcctggctaacatggcaaaaccccatctctactaaaaatacaaaaattagccggacgtggtggtgcctgcctgtaatccagctactcaggaggctgaggcaggataattttgcttaaacccgggaggcggaggttgcagtgagccgagatcacaccactgcactccaacctgggaaacgcagtgagactgtgcctcaaaaaaaagaaaggaagaaaaaagaaaactcaggaaacagatcttgggggacactccagggaacccaaaactcaaaggcggagagctcagtgggcaccaccaaggcgagatgaagccccagcaggcaccttcagaagacccacgtagactgcagaccctgccacggacaatactaaggacaaaaacccagagacttggggtctgtgggcccccaaacatggggtaaagttgatttgcctgatattcaggaagaaggggtgaggggtgggtatttatgcttttgattcagaagaaagtggggcttgggattccagggacttggctgggggtgggaaacttcatccacttccctactctcatcatgagtacggacagggtgggcgggagactgatcatcgggactcatcatgaagagcccagccccaccccacatactcagatcccacccacagactggtggccacacctcagcctggtcacaaagagttacactcagatacatgagcacggcagcgtgctcataactgtttaacaaccagctgtcctgggagggggacagctttgtaatgtttgccaatttccatggtgtaaatgctaccaccatggctgatttcatcactgccaagcatagacatccctaataggacaccacggatctgtccccggcatccggcccagggcctggcacaaagcatgctctagggaaatgcttgctgattgaaaggaaggaagaatgactctacagtcacacctatggcatcccacaaaatctgtcacatggctgcataatctcagccactctttcacaactatagactcatacacgcgaagtgccagattcatgcacaaccacacaatcacatggaagtcacagacggcatcacagacagtcacagcactgtgtgtatgttataacacaagcacacaaaactcagacagcatcccagctacacagccactcccagaggtgtcaccgtcacacttggtaattaatactcattacattagacacagacagaccaagttatagtcagacctggttacacacatacacacacacaatatcaccatgacaaatacacattacacacacacaacatcacaatgacaaacacacattacacacacaacatcacgatgacaaacacacattacacacacaacatcacgatgacaaacacacattacacacacatcacaatgacaaacacaacattacacacacacaacatcacaatgacacacacatcacacacacatcacaatgacaaacacacaacattacacacatatacacacagcctgagggccctccccagcccagactaacacatctcggggtgaggaccagaccttgttcataaccctgggcctcttaaccactgatctttgaaataaatggcaaatagttgtacctgga

(19) United States
(12) Patent Application Publication
(10) Pub. No.: US 2018/0369114 A1
(43) Pub. Date: Dec. 27, 2018

(54) COMPOSITION FOR SKIN WHITENING COMRPISING TNFSF14 PROTEIN

(71) Applicant: AMOREPACIFIC CORPORATION, SEOUL (KR)

(72) Inventors: Kyu-Han KIM, Yongin-si (KR); Hyoung-June KIM, Yongin-si (KR); Tae Ryong LEE, Yongin-si (KR)

(21) Appl. No.: 16/014,677

(22) Filed: Jun. 21, 2018

(30) Foreign Application Priority Data
Jun. 27, 2017 (KR) 10-2017-0081207

(51) Int. Cl. A61K 8/64; A61Q 5/08; A61Q 19/02

(52) U.S. Cl. CPC A61K 8/64; A61Q 19/02; A61Q 5/08

(57) ABSTRACT

The present specification is intended to develop a new substance which exhibits a skin whitening effect and to apply the new substance to a composition for skin whitening, provides a new composition for skin whitening and a new kit for skin whitening which contain TNFSF14 protein and a polynucleotide encoding the TNFSF14 protein and a method of using the same, and thus can contribute to the market expansion and industry development related to the skin whitening field.

This application claims priority to Korean Patent Application No. 10-2017-0081207, filed on Jun. 27, 2017, and all the benefits accruing therefrom under 35 U.S.C. §119, the content of which in its entirety is herein incorporated by reference.

### BACKGROUND

### Field of the Invention

The present specification relates to a composition for skin whitening containing TNFSF14 protein.

### Background Art

Melanin is a biopolymer material of a phenol having a complex form of a black pigment and proteins and is observed when cut surfaces of apples, potatoes, and bananas are exposed to air and thus browned, or observed from outer feathers, skin, head, eyes, and the like of animals. Melanin is directly associated with skin whitening and the like since it is deposited on the skin to form melasmas, freckles, and the like when being overproduced, and melanin promotes skin aging and may also cause skin cancer.

Melanocyte stimulating hormone (MSH) is secreted by ultraviolet light, inflammation, hormone, and the like. MSH reacts with a receptor to enhance cAMP in melanocytes and thus to synthesize melanin, and the melanin synthesized is secreted to the outside of melanocytes and plays a role to protect the skin from ultraviolet light and the like. The synthesis of melanin is mainly regulated by α-MSH, and MITF, TYR, TRP1, TRP2, and the like are known as the proteins to be involved in the synthesis of melanin.

Tumor necrosis factor superfamily member 14 (TNFSF14) protein is a ligand for tumor necrosis factor receptor superfamily 14 (TNFSF14). This protein serves as an auxiliary stimulus for the activity of lymphocytes and may also function to inhibit the infection by herpes virus. It is also known that this protein stimulates proliferation of T cells and causes apoptosis of tumor cells.

However, the research on that TNFSF14 protein is involved in skin whitening effect or melanin production has not yet been known.

### CITATION LIST

### Patent Literature

Patent Literature 1: Korean Patent No. 10-1735564 (May 08, 2017)

### SUMMARY

### Technical Problem

In an aspect, an object of the present invention is to develop a new substance which exhibits a skin whitening effect and to apply this new substance to a composition for skin whitening.

### Solution to Problem

In an aspect, the present invention provides a composition for skin whitening, which comprises one or more of tumor necrosis factor superfamily member 14 (TNFSF14) protein, a protein having 90% or more amino acid sequence homology with the TNFSF14 protein, and a polynucleotide encoding the proteins.

In another aspect, the present invention provides a method for skin whitening, comprising administering a composition comprising one or more of tumor necrosis factor superfamily member 14 (TNFSF14) protein, a protein having 90% or more amino acid sequence homology with the TNFSF14 protein, and a polynucleotide encoding the proteins to a subject in need thereof.

In another aspect, the present invention provides a composition for skin whitening, which comprises a substance enhancing the expression of TNFSF14 protein.

In another aspect, the present invention provides a method for skin whitening, comprising administering a composition comprising a substance enhancing the expression of TNFSF14 protein to a subject in need thereof.

In another aspect, the present invention provides a composition for body hair color control, which comprises one or more of TNFSF14 protein, a protein having 90% or more amino acid sequence homology with the TNFSF14 protein, and a polynucleotide encoding the proteins.

In another aspect, the present invention provides a method of controlling body hair color for the purpose of beauty, which comprises treating body hair with the composition.

In still another aspect, the present invention provides a method of screening a substance having a skin whitening effect, comprising: treating a cell with a test substance; and confirming whether an expression level of TNFSF14 protein or a polynucleotide encoding the TNFSF14 protein in the cell is changed or not after the above step.

In still another aspect, the present invention provides a kit for screening a substance having a skin whitening effect, comprising a measurement unit for displaying relative degrees of expression of TNFSF14 protein or a polynucleotide encoding the TNFSF14 protein before and after being treated with a test substance.

In yet another aspect, the present invention provides a method of providing information required for skin condition diagnosis, comprising confirming an expression level of TNFSF14 protein or a polynucleotide encoding the TNFSF14 protein in an individual.

### Advantageous Effects of Invention

The present invention provides a new composition for skin whitening, which comprises TNFSF14 protein, a protein having 90% or more amino acid sequence homology with the TNFSF14 protein, or a polynucleotide encoding the proteins and a method of using the same in an aspect, and thus it can contribute to the market expansion and industry development related to the skin whitening field.

### BRIEF DESCRIPTION OF DRAWINGS

FIG. 1 illustrates the results on the change in color when melanocytes are treated with TNFSF14 protein;

FIG. 2 illustrates the relative amounts of melanin produced when melanocytes treated with TNFSF14 protein are compared with melanocytes untreated with TNFSF14 protein; and

FIG. 3 illustrates the relative amounts of melanin produced when melanocytes are treated with TNFSF14 protein at different concentrations.

### DETAILED DESCRIPTION

Hereinafter, the present specification will be described in detail.

As used herein, the term “skin” means a tissue which covers the body surface of an animal and is the widest concept including not only a tissue which covers the body surface of the face, the body, or the like but also scalp and hair.

As used herein, “TNFSF14” refers to the tumor necrosis factor superfamily member 14 and is also mentioned as LIGHT (lymphotoxins, exhibits inducible expression, and competes with HSV glycoprotein D for HVEM, a receptor expressed by T lymphocytes).

As used herein, “recombinant protein” is also called a gene-recombinant protein and refers to a protein obtained by treating one or more polynucleotides or genes by recombinant techniques and then encoding a protein by the one or more polynucleotides or genes treated.

TNFSF14 protein is associated with tumor necrosis factor receptors and is generally a protein which has been the subject of tumor-related studies or inflammation-related studies. However, it has not been known at all whether the TNFSF14 protein and genes related thereto exhibit skin-related effects, in particular, skin whitening and body hair color control effects or not.

The present specification has experimentally investigated that TNFSF14 protein, a protein having 90% or more amino acid sequence homology with the TNFSF14 protein, and a polynucleotide encoding the proteins inhibit the melanin production of melanocytes and has demonstrated that the proteins and the polynucleotide can be used in a composition for skin whitening, a composition for body hair color control, a method of controlling body hair color, a method of screening a substance having a skin whitening effect, a kit for screening a substance having a skin whitening effect, a method of diagnosing a skin condition, a method of providing information required for skin condition diagnosis, or the like.

In an aspect, the present invention relates to a composition for skin whitening, which comprises one or more of tumor necrosis factor superfamily member 14 (TNFSF14) protein, a protein having 90% or more amino acid sequence homology with the TNFSF14 protein, or a polynucleotide encoding the proteins.

The protein having 90% or more amino acid sequence homology with the TNFSF14 protein, that is disclosed herein, may include proteins having 80% or more, 85% or more, 90% or more, 95% or more, 96% or more, 97% or more, 98% or more, and 99% or more amino acid sequence homology with TNFSF14 protein. In addition, the protein disclosed herein may include a protein including SEQ ID NO: 1 or a protein in which fragments of SEQ ID NO: 1 and one or more amino acids, two or more amino acids, three or more amino acids, four or more amino acids, five or more amino acids, six or more amino acids, or seven or more amino acids are changed.

In an aspect of the present invention, the protein may be contained in the composition in the form of being conjugated with a labeling substance. According to another aspect, the labeling substance may be a fluorescent substance or a contrasting substance. In another aspect of the present invention, the fluorescent substance may be fluorescein isothiocyanate (FITC).

In an aspect of the present invention, the amino acid change belongs to properties which cause a change in physicochemical properties of peptides or proteins. For example, amino acid changes that the thermal stability of peptides is improved, the substrate specificity is altered, the optimum pH is changed, and the like, can be performed.

As used herein, the term “amino acid” includes not only the 22 standard amino acids which are naturally incorporated into peptides or proteins but also D-isomers and modified amino acids. Accordingly, in an aspect of the present invention, peptides may be peptides including D-amino acid. Meanwhile, in another aspect of the present invention, peptides or proteins may include non-standard amino acids which have been post-translationally modified, and the like. Examples of the post-translational modification may include phosphorylation, glycosylation, acylation (including, for example, acetylation, myristoylation, and palmitoylation), alkylation, carboxylation, hydroxylation, glycation, biotinylation, ubiquitinylation, changes in chemical properties (for example, beta-elimination deimidization and deamidization), and structural changes (for example, formation of a disulfide bridge). In addition, examples thereof may include amino acid changes caused by chemical reactions occurring in the course of binding with crosslinkers for forming peptide conjugates, for example, amino acid changes such as changes in amino groups, carboxyl groups, or another side chains.

The proteins or peptides disclosed herein may be wild-type proteins or peptides identified and isolated from natural sources. Meanwhile, the proteins or peptides disclosed herein may be a variant including an amino acid sequence in which one or more amino acids are substituted, deleted and/or inserted as compared with the entire amino acid sequence of SEQ ID NO: 1 or a fragment thereof. Amino acid changes not only in variants but also in wild-type proteins or wild-type polypeptides include conservative amino acid substitutions which do not significantly affect protein folding and/or activity. Examples of conservative substitutions are within the family of basic amino acids (arginine, lysine, and histidine), acidic amino acids (glutamic and aspartic acid), polar amino acids (glutamine and asparagine), hydrophobic amino acids (leucine, isoleucine, valine, and methionine), aromatic amino acids (phenylalanine, tryptophan, and tyrosine), and small amino acids (glycine, alanine, serine, and threonine). In general, amino acid substitutions which do not alter specific activity are known in the art. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly and vice versa. Other examples of conservative substitutions include those presented in the following table.

TABLE 1

| Original | Exemplary residue | Preferred residue |
|---|---|---|
| amino acid | substitution | substitution |
| Ala (A) | val; leu; ile | Val |
| Arg (R) | lys; gln; asn | Lys |
| Asn (N) | gln; his; asp, lys; | Gln |
| | arg | |
| Asp (D) | glu; asn | Glu |
| Cys (C) | ser; ala | Ser |
| Gln (Q) | asn; glu | Asn |
| Glu (E) | asp; gln | Asp |
| Gly (G) | Ala | Ala |
| His (H) | asn; gln; lys; arg | Arg |
| Ile (I) | leu; val; met; ala; | Leu |
| | phe; norleucine | |
| Leu (L) | norleucine; ile; val; | Ile |
| | met; ala; phe | |
| Lys (K) | arg; gln; asn | Arg |
| Met (M) | leu; phe; ile | Leu |
| Phe (F) | leu; val; ile; ala; | Tyr |
| | tyr | |
| Pro (P) | Ala | Ala |
| Ser (S) | thr | Thr |
| Thr (T) | Ser | Ser |
| Trp (W) | tyr; phe | Tyr |
| Tyr (Y) | trp; phe; thr; ser | Phe |
| Val (V) | ile; leu; met; phe; | Leu |
| | ala; norleucine | |

Substantial modification in the biological properties of proteins or peptides is performed by choosing substituents which significantly differ in (a) their effect for maintaining the structure of the polypeptide backbone within the substitution region, for example, the sheet or helical conformation, (b) their effect for maintaining the charge or hydrophobicity of the molecule at the target site, or (c) their effect for maintaining the bulk of the side chain. The natural residues are classified into the following groups based on the usual side chain properties:

(1) Hydrophobic residues: norleucine, met, ala, val, leu, and ile;

(2) Neutral hydrophilic residues: cys, ser, and thr;

(3) Acidic residues: asp and glu;

(4) Basic residues: asn, gln, his, lys, and arg;

(5) Residues affecting chain orientation: gly and pro; and

(6) Aromatic residues: trp, tyr, and phe.

Non-conservative substitutions will occur by exchanging one member of these classes for another class. Any cysteine residue, which is not associated with maintenance of the proper stereostructure of proteins or peptides, can also be generally substituted with serine to enhance the oxidative stability of the molecule and to prevent strange crosslinking. In other words, cysteine bond(s) can be added to the proteins or peptides to improve the stability.

Other types of amino acid variants of proteins or peptides are those in which the glycosylation pattern of antibody is changed. The term change refers to the deletion of one or more carbohydrate moieties found in the peptide and/or the addition of one or more glycosylation sites which are not present in the protein or peptide.

The glycosylation of proteins or peptides is typically N-linking or O-linking. N-linking means that the carbohydrate moiety is attached to the side chain of the asparagine residue. The tripeptide sequences asparagine-X-serine and asparagine-X-threonine (where X is an arbitrary amino acid except proline) are recognition sequences for enzymatically attaching carbohydrate moieties to asparagine side chains. Hence, a potential glycosylation site is created when one of these tripeptide sequences is present in the polypeptide. O-linked glycosylation means to attach one of sugar N-acetylgalactosamine, galactose, or xylose to a hydroxyamino acid, most commonly serine or threonine, but 5-hydroxyproline or 5-hydroxylysine may also be used.

Addition of a glycosylation site to a protein or peptide is conveniently performed by changing the amino acid sequence so as to contain one or more of the above-mentioned tripeptide sequences (in the case of N-linked glycosylation sites). Such changes may be achieved by adding one or more serine or threonine residues to the sequence of the original antibody or substituting the sequence of the original antibody with these residues (in the case of O-linked glycosylation sites).

As an embodiment, the TNFSF14 protein may have or comprise the amino acid sequence represented by SEQ ID NO: 1. In another aspect of the present invention, a protein having the sequence of SEQ ID NO: 1, a peptide which is a fragment of the sequence of SEQ ID NO: 1, and proteins or peptides having 90% or more sequence homology with the protein or peptide sequence include those that are synthesized or recombinantly produced.

As another embodiment, the polynucleotide may have or comprise a base sequence encoding the amino acid sequence represented by SEQ ID NO: 1.

As another embodiment, the polynucleotide may be mRNA of TNFSF14 gene.

As another embodiment, the composition may inhibit melanin production or decrease melanin expression. Melanin is observed from outer feathers, skin, head, eyes, and the like of animals. When melanin is overproduced, it is deposited on the skin to form melasmas, freckles, and the like, and also promotes skin aging, and may also cause skin cancer. The disease or symptom due to the melanin overproduction is known as one or more selected from the group consisting of spots, freckles, age spots, blemishes, epidermal melanocytic lesions, cafe's au lait macules, nevi, Becker's nevus, nevus spilus, lentigines, lentigo, dermal melanocytic lesions, Mongolian spot, nevus of Ota, acquired bilateral nevus of Ota-like macules, nevus of Ito, blue nevus, melanocytic nevus, junctional nevus, compound nevus, intradermal nevus, halo nevus, congenital nevocytic nevus, Spitz nevus, dysplastic nevus, melanoma, lentigo maligna melanoma, superficial spreading melanoma, acral lentiginous melanoma, nodular melanoma, pigment basal cell carcinoma, dermatofibromas, dermoid cyst, keloid, pigmentation by ultraviolet light, pigmentation by drug, pigmentation after inflammation, pigmentation by dermatitis, and keratoacanthomas. TNFSF14 protein and a polynucleotide encoding the TNFSF14 protein according to an aspect of the present invention can inhibit melanogenesis in cells, the disease or symptom described above can be improved when the melanin production is inhibited, and thus the present invention can improve the disease or symptom described above in an aspect (see Experimental Example 1).

As another embodiment, the whitening is to improve or alleviate one or more selected from the group consisting of melasmas, freckles, lentigo, nevi, melanoma, pigmentation by ultraviolet light, pigmentation by drug, pigmentation after inflammation, and pigmentation by dermatitis.

As another embodiment, the content of the protein may be from 0.00001 to 10 wt % with respect to the total weight of the composition. In an aspect, the content may be 0.00001 wt % or more, 0.00005 wt % or more, 0.0001 wt % or more, 0.0005 wt % or more, 0.001 wt % or more, 0.005 wt % or more, 0.01 wt % or more, 0.05 wt % or more, 0.1 wt % or more, 0.5 wt % or more, 1 wt % or more, 3 wt % or more, 5 wt % or more, or 8 wt % or more. In another aspect, the content may be 10 wt % or less, 8 wt % or less, 5 wt % or less, 3 wt % or less, 2 wt % or less, 1 wt % or less, 0.5 wt % or less, 0.1 wt % or less, 0.05 wt % or less, 0.01 wt % or less, 0.005 wt % or less, 0.001 wt % or less, 0.0005 wt % or less, 0.0001 wt % or less, 0.00005 wt % or less, or 0.00003 wt % or less.

As another embodiment, the dosage of the protein may be from 0.00001 mg/kg/day to 20 mg/kg/day. In an aspect, the dosage may be 0.00001 mg/kg/day or more, 0.00005 mg/kg/day or more, 0.0001 mg/kg/day or more, 0.0005 mg/kg/day or more, 0.001 mg/kg/day or more, 0.005 mg/kg/day or more, 0.01 mg/kg/day or more, 0.05 mg/kg/day or more, 0.1 mg/kg/day or more, 0.5 mg/kg/day or more, 1 mg/kg/day or more, 3 mg/kg/day or more, 5 mg/kg/day or more, 8 mg/kg/day or more, 10 mg/kg/day or more, 14 mg/kg/day or more, 16 mg/kg/day or more, or 18 mg/kg/day or more. In another aspect, the dosage may be 20 mg/kg/day or less, 18 mg/kg/day or less, 16 mg/kg/day or less, 14 mg/kg/day or less, 12 mg/kg/day or less, 10 mg/kg/day or less, 8 mg/kg/day or less, 6 mg/kg/day or less, 4 mg/kg/day or less, 2 mg/kg/day or less, 1 mg/kg/day or less, 0.5 mg/kg/day or less, 0.1 mg/kg/day or less, 0.05 mg/kg/day or less, 0.01 mg/kg/day or less, 0.005 mg/kg/day or less, 0.001 mg/kg/day or less, 0.0005 mg/kg/day or less, 0.0001 mg/kg/day or less, or 0.00005 mg/kg/day or less. The determination of the dosage of the ingredient is within the level of those skilled in the art, and the daily dosage may vary depending on various factors such as the progress of the subject to be administered, the time of onset, age, health condition, complications, and the like.

As an embodiment, the administration route of the composition may be transdermal, subcutaneous, intradermal, intramuscular, intravascular, or oral administration, but it is not limited thereto, and transdermal administration is preferable.

As another embodiment, the polynucleotide may be administered by being contained in a vector. As the vector, any vector can be used as long as it can be used by those skilled in the art and can contain the polynucleotide.

As another embodiment, the composition for skin whitening may be a cosmetic composition.

In another aspect, the present invention may be a composition for skin whitening, which comprises a substance which enhances the expression of tumor necrosis factor superfamily member 14 (TNFSF14) protein. When TNFSF14 protein expression is enhanced, the melanin content in melanocytes is decreased by an increased amount of TNFSF14 protein and thus a substance which enhances TNFSF14 protein expression can be used in a composition for skin whitening.

In another aspect, the present invention may be a composition for body hair color control, which comprises one or more of tumor necrosis factor superfamily member 14 (TNFSF14) protein, a protein having 90% or more amino acid sequence homology with the TNFSF14 protein, or a polynucleotide encoding the proteins.

As an embodiment, the body hair includes all the hairs of the body, but it may preferably be hair.

As another embodiment, the color control may be one or more selected from the group consisting of whitening, graying, browning, yellowing, and turning red.

In another aspect, the present invention may be a kit for skin whitening, which comprises the composition and the instructions.

In another aspect, the present invention may be a kit for hair color control, which comprises the composition and the instructions.

In an aspect, the present invention may be a method of controlling body hair color for the purpose of beauty, comprising treating body hair with the composition according to an aspect of the present invention.

In another aspect, the present invention may be a method of screening a substance having a skin whitening effect, comprising:

treating a cell with a test substance; and

confirming whether an expression level of tumor necrosis factor superfamily member 14 (TNFSF14) protein or a polynucleotide encoding the TNFSF14 protein in the cell is changed or not after the above step.

As an embodiment, the present invention may be a method of screening a substance having a skin whitening effect, further comprising judging the test substance as a substance having a skin whitening effect when an expression level of TNFSF14 protein or a polynucleotide encoding the TNFSF14 protein after being treated with the test substance is higher than an expression level of the TNFSF14 protein or the polynucleotide encoding the TNFSF14 protein before being treated with the test substance.

In another aspect, the present invention may be a kit for screening a substance having a skin whitening effect, comprising a measurement unit for displaying relative degrees of expression of tumor necrosis factor superfamily member 14 (TNFSF14) protein or a polynucleotide encoding the TNFSF14 protein before and after being treated with a test substance.

As an embodiment, the kit may further comprise melanocytes and instructions, and the method of screening a substance having a skin whitening effect may be described in the instructions.

In another aspect, the present invention may be a method of providing information required for skin condition diagnosis, comprising confirming an expression level of tumor necrosis factor superfamily member 14 (TNFSF14) protein or a polynucleotide encoding the TNFSF14 protein in an individual.

As an embodiment, the skin condition may be a skin condition associated with melanin.

As used herein, the “skin condition associated with melanin” means a skin condition that is directly or indirectly related to a melanin pigment or melanocyte, and for example, the skin condition includes skin which exhibits high or low sensitivity to a stimulus to form melanin, skin which has a possibility that deposition of melanin will further proceed or be inhibited in the future, skin which is at high or low risk of skin disease associated with melanin, and skin which has a dark or light tone.

According to an aspect of the present invention, the stimulus may be one or more of an extrinsic stimulus or an endogenous stimulus.

According to an embodiment of the present invention, the extrinsic stimulus may include stimuli by ultraviolet light, physical pressure, scars, and a heavy metal, a stimulus by a chemical substance, infection by microorganisms, and stress.

According to another embodiment of the present invention, the endogenous stimulus may include an oxidative stress change in the body, aging, inflammation, a hormonal change, digestive organ dysfunction including liver, a gene abnormality, a metabolic disorder, a malnutrition condition, and excess or deficiency of vitamins and minerals.

As another embodiment, the confirmation of expression level may be to compare the degrees of expression of TNFSF14 protein or a polynucleotide encoding the TNFSF14 protein before and after a specific stimulus to form melanin is applied to the TNFSF14 protein or the polynucleotide encoding the TNFSF14 protein.

In an embodiment, the confirmation of expression level or the comparison of degrees of expression may be appropriately selected from known techniques, for example, immunofluorescence analysis, western blot, dot blot, ELISA, northern blot, PCR, RT-qPCR, GC-MS, LC-MS, NMR, and the like by those skilled in the art, but it is not limited thereto.

For example, the confirmation of expression level or the comparison of degrees of expression may be to directly measure degrees of expression of TNFSF14 protein or a polynucleotide encoding the TNFSF14 protein from an individual with harvesting melanocytes or without isolating the cells to compare the degrees of expression with each other. The harvesting or measurement can be performed by any method known to those skilled in the art.

According to another embodiment of the present invention, the confirmation of expression level or the comparison of degrees of expression of the TNFSF14 protein or the polynucleotide encoding the TNFSF14 protein may be to compare the expression levels or the degrees of expression of TNFSF14 protein or the polynucleotide encoding the TNFSF14 protein in the entire skin or a specific skin area of an individual at certain two time points. For example, it may be to directly measure expression levels or degrees of expression of TNFSF14 protein or a polynucleotide encoding the TNFSF14 protein from an individual with harvesting melanocytes or without isolating the cells from the entire skin or a specific skin area to compare the expression levels or degrees of expression with each other. The harvesting or measurement can be performed by any method known in the art.

As another embodiment, the skin condition diagnosis may be to judge skin as skin in which melanin deposition is increased or decreased in the entire skin or a specific skin area and thus skin blackening or whitening possibly proceeds. As used herein, “skin blackening” includes a phenomenon in which various factors or stimuli are applied or eliminated and the entire skin or a specific skin area thus changes dark or black. In addition, as used herein, “skin whitening” includes a phenomenon in which various factors or stimuli are applied or eliminated and the entire skin or a specific skin area thus changes bright or white.

According to another aspect of the present invention, the skin condition diagnosis may be to judge the grade of skin blackening or whitening probability according to the degree of expression of TNFSF14 protein or a polynucleotide encoding the TNFSF14 protein.

According to an embodiment, the grade may be a reference value determined based on the data on the degree of expression of TNFSF14 protein or a polynucleotide encoding the TNFSF14 protein obtained from skin samples prepared using a certain number of individuals and a grade determined by dividing the section based on the reference value.

When the composition is a pharmaceutical composition, the pharmaceutical composition may additionally contain a preservative, an antiseptic, a stabilizer, a wetting or emulsifying accelerator, a pharmaceutical adjuvant such as a salt and/or buffer for controlling osmotic pressure, and other therapeutically useful substances, and the pharmaceutical composition may be formulated in various oral or parenteral administration forms according to conventional methods.

When the administration route of the composition is oral administration, the composition may take formulations such as tablets, pills, hard and soft capsules, liquids, suspensions, emulsions, syrups, powders, powder remedies, fine granules, granules, pellets, and the like, and such formulations may contain, a surfactant, a diluent (for example, lactose, dextrose, sucrose, mannitol, sorbitol, cellulose, or glycine), and a lubricant (for example, silica, talc, stearic acid and magnesium or calcium salt thereof or polyethylene glycol) in addition to the active ingredient. The tablets may also contain a binder such as magnesium aluminum silicate, starch paste, gelatin, tragacanth, methylcellulose, sodium carboxymethylcellulose, or polyvinylpyrrolidine, and the tablets may contain pharmaceutical additives such as a disintegrating agent such as starch, agar, and alginic acid or sodium salt thereof, an absorbing agent, a coloring agent, a flavoring agent, and a sweetening agent if necessary. The tablets may be prepared by conventional mixing, granulating, or coating methods.

In addition, when the administration route of the composition is parenteral administration, and the form of parenteral administration may be a formulation of transdermal administration, and for example, the formulation may be formulations such as injections, drops, ointments, lotions, gels, creams, sprays, suspensions, emulsions, suppositories, patches, and the like, but it is not limited thereto.

The pharmaceutical composition may be administered parenterally, rectally, topically, transdermally, subcutaneously, and the like.

The pharmaceutical composition may be an external preparation for skin, and the external preparation for skin is a generic term that may include anything to be applied from the outside of the skin, and various formulations of medicines or quasi-drugs may be included therein.

According to still another aspect of the present invention, the composition may be a composition for skin whitening, which is a cosmetic composition.

The cosmetic composition may additionally contain functional additives and ingredients to be contained in general cosmetic compositions. The functional additives may include ingredients selected from the group consisting of water-soluble vitamins, oil-soluble vitamins, polymeric peptides, polymeric polysaccharides, sphingolipids, and seaweed extracts. Ingredients to be contained other than these may include oil and fat ingredients, a moisturizer, an emollient, a surfactant, organic and inorganic pigments, an organic powder, an ultraviolet absorber, an antiseptic, a bactericide, an antioxidant, a plant extract, a pH adjusting agent, an alcohol, a colorant, a perfume, a blood circulation accelerator, a cold feel providing agent, an antiperspirant agent, purified water, and the like.

The formulation of the cosmetic composition is not particularly limited and can be appropriately selected depending on the purpose. For example, the cosmetic composition may be prepared as one or more formulations selected from the group consisting of skin lotion, skin softener, skin toner, astringent, lotion, milky lotion, moisturizing lotion, nourishing lotion, massage cream, nourishing cream, moisturizing cream, hand cream, foundation, essence, nourishing essence, pack, soap, cleansing foam, cleansing lotion, cleansing cream, body lotion, and body cleanser, but it is not limited thereto.

When the formulation according to an aspect of the present invention is a paste, a cream, or a gel, an animal fiber, a plant fiber, wax, paraffin, starch, tragacanth, a cellulose derivative, polyethylene glycol, silicone, bentonite, silica, talc, zinc oxide, or the like may be used as a carrier component.

When the formulation according to an aspect of the present invention is a powder or a spray, lactose, talc, silica, aluminum hydroxide, calcium silicate, or a polyamide powder may be used as a carrier component, and particularly in the case of a spray, it may contain a propellant such as chlorofluorohydrocarbon, propane/butane, or dimethyl ether.

When the formulation according to an aspect of the present invention is a solution or an emulsion, a solvent, a solvating agent, or an emulsifier is used as a carrier component, and for example, there is water, ethanol, isopropanol, ethyl carbonate, ethyl acetate, benzyl alcohol, benzyl benzoate, propylene glycol, 1,3-butyl glycol oil, glycerol aliphatic ester, polyethylene glycol, or fatty acid esters of sorbitan.

When the formulation according to an aspect of the present invention is a suspension, a liquid diluent such as water, ethanol, or propylene glycol, a suspending agent such as ethoxylated isostearyl alcohol, polyoxyethylene sorbitol ester, or polyoxyethylene sorbitan ester, microcrystalline cellulose, aluminum metahydroxide, bentonite, agar, tragacanth, or the like may be used as a carrier component.

When the formulation according to an aspect of the present invention is a surfactant-containing cleanser, an aliphatic alcohol sulfate, an aliphatic alcohol ether sulfate, a sulfosuccinic acid monoester, an isethionate, an imidazolinium derivative, methyl taurate, a sarcosinate, a fatty acid amide ether sulfate, an alkylamido betaine, an aliphatic alcohol, a fatty acid glyceride, a fatty acid diethanolamide, vegetable oils, a linolenic derivative, an ethoxylated glycerol fatty acid ester, or the like may be used as a carrier component.

According to another aspect of the present invention, the composition may be a composition for skin whitening which is a food or health food composition.

The food or health food composition may be a formulation in a liquid or solid form, for example, there are various kinds of food, beverages, gum, tea, vitamin complexes, health functional food, health supplement food, and the like, and it may be used in the form of powders, granules, tablets, capsules, or beverages. In the respective formulations of the food composition, ingredients to be commonly used in the field other than the active ingredient may be appropriately chosen and blended depending on the formulation or the purpose of use by those skilled in the art without difficulty, and synergistic effects may be exhibited when the active ingredient is simultaneously applied with other ingredients.

There are no particular limitations on the liquid ingredients which may be contained other than the active ingredient disclosed herein, and various flavoring agents or natural carbohydrates may be added as additional ingredients as in ordinary beverages. Examples of the natural carbohydrates may include monosaccharides, disaccharides such as glucose and fructose, polysaccharides such as maltose and sucrose, common saccharides such as dextrin and cyclodextrin, and sugar alcohols such as xylitol, sorbitol, and erythritol. Natural flavoring agents (thaumatin, stevia extracts (for example, rebaudioside A and glycyrrhizin) and synthetic flavoring agents (for example, saccharin and aspartame) may be advantageously used as the flavoring agents. The ratio of the natural carbohydrates may be generally about from 1 to 20g per 100 ml or 100 g of the composition disclosed herein, and it may be about from 5 to 12 g per 100 ml or 100 g of the composition in an aspect.

In an aspect, the food composition may contain various nutrients, vitamins, minerals (electrolytes), flavors such as synthetic flavors, natural flavors, and the like, coloring agents, and thickening agents (cheese, chocolate, and the like), pectic acid and any salt thereof, alginic acid and any salt thereof, an organic acid, a protective colloid thickener, a pH adjusting agent, a stabilizer, an antiseptic, glycerin, an alcohol, a carbonating agent to be used in a carbonated beverage, and the like. In another aspect, the food composition may contain flesh for the production of natural fruit juices and vegetable beverages. These ingredients may be used independently or in combination. The ratio of the additives may vary, but it is generally selected in a range of from 0.001 to about 20 parts by weight per 100 parts by weight of the composition disclosed herein.

The relationship between skin pigmentation and TNFSF14 protein or a polynucleotide encoding the TNFSF14 protein has not been known up to date, and the present invention has identified such a relationship in an aspect. The present invention has found a method of screening a substance exerting a skin whitening effect by using this relationship. In addition, it is possible to manufacture a kit comprising a measurement unit capable of confirming or displaying the degree of expression of TNFSF14 protein or a polynucleotide encoding the TNFSF14 protein by applying this point. Moreover, it is possible to provide information required for evaluating or diagnosing the skin condition associated with melanin by confirming the degree of expression of TNFSF14 protein or a polynucleotide encoding the TNFSF14 protein.

Hereinafter, the configuration and effect of an aspect of the present invention will be described in more detail with reference to Examples and Experimental Examples. However, the following examples are provided for illustrative purposes only to facilitate understanding of the present invention, and the gist and scope of the present invention are not limited thereto.

### EXAMPLE 1

### Preparation of Experimental Materials

Tumor necrosis factor superfamily member 14 (TNFSF14) is a ligand gene, and experiments on the protein encoded by the gene are generally performed using a recombinant protein. A recombinant protein of TNFSF14 was obtained to confirm the use and effect according to an aspect of the present invention.

Specifically, recombinant human LIGHT/TNFSF14 protein was purchased (catalog number 664-LI, Genbank accession No. 043557) from R&D systems (614 McKinley Place NE, Minneapolis, Minn. 55413, US).

The amino acid sequence of the recombinant human TNFSF14 protein is as follows.

SEQ ID NO: 1
MEESVVRPSVFVVDGQTDIPFTRLGRSHRRQSCSVARVGLGLLLLLMGAG
LAVQGWFLLQLHWRLGEMVTRLPDGPAGSWEQLIQERRSHEVNPAAHLTG
ANSSLTGSGGPLLWETQLGLAFLRGLSYHDGALVVTKAGYYYIYSKVQLG
GVGCPLGLASTITHGLYKRTPRYPEELELLVSQQSPCGRATSSSRVWWDS
SFLGGVVHLEAGEKVVVRVLDERLVRLRDGTRSYFGAFMV

The base sequence (or mRNA base sequence) of TNFSF14 gene is composed of the base corresponding to the amino acid sequence of SEQ ID NO: 1 and is the same as the base sequence of the following SEQ ID NO: 2 (or Genbank accession number NM_003807).

SEQ ID NO: 2
CGAGACTCCATCTCAAAAACAAAACAAATAAACGAACAAAAAAACCCACA
ACGTATTATTTTCTTGTTTACGAGGTTTCTTGTCTCTCTGGCTCCACCAG
AAGAGGAGCAGGGACCCTTCTTGCTGTTGTTCATTGCTGCATCCCCCACA
CCGAGAGCAGAGCCTGGCATGGGCAGAAAGTCCTCAGTCGATATTTGGTG
GCCCCAAGCGAATGAAGCATCCAAGAAGGGAAAGCTGGGGGCTCCCCACT
GCACTTGCCACCTGAGTCACATTTTCAGAAGCCTCTGGAAAGTCGTGCAC
AGCCCAGGAGTGTTGAGCAATTTCGGTTTCCTCTGAGGTTGAAGGACCCA
GGCGTGTCAGCCCTGCTCCAGACACCTTGGGCATGGAGGAGAGTGTCGTA
CGGCCCTCAGTGTTTGTGGTGGATGGACAGACCGACATCCCATTCACGAG
GCTGGGACGAAGCCACCGGAGACAGTCGTGCAGTGTGGCCCGGGTGGGTC
TGGGTCTCTTGCTGTTGCTGATGGGGGCCGGGCTGGCCGTCCAAGGCTGG
TTCCTCCTGCAGCTGCACTGGCGTCTAGGAGAGATGGTCACCCGCCTGCC
TGACGGACCTGCAGGCTCCTGGGAGCAGCTGATACAAGAGCGAAGGTCTC
ACGAGGTCAACCCAGCAGCGCATCTCACAGGGGCCAACTCCAGCTTGACC
GGCAGCGGGGGGCCGCTGTTATGGGAGACTCAGCTGGGCCTGGCCTTCCT
GAGGGGCCTCAGCTACCACGATGGGGCCCTTGTGGTCACCAAAGCTGGCT
ACTACTACATCTACTCCAAGGTGCAGCTGGGCGGTGTGGGCTGCCCGCTG
GGCCTGGCCAGCACCATCACCCACGGCCTCTACAAGCGCACACCCCGCTA
CCCCGAGGAGCTGGAGCTGTTGGTCAGCCAGCAGTCACCCTGCGGACGGG
CCACCAGCAGCTCCCGGGTCTGGTGGGACAGCAGCTTCCTGGGTGGTGTG
GTACACCTGGAGGCTGGGGAGAAGGTGGTCGTCCGTGTGCTGGATGAACG
CCTGGTTCGACTGCGTGATGGTACCCGGTCTTACTTCGGGCCTTTCATGG
TGTGAAGGAAGGAGCGTGGTGCATTGGACATGGGTCTGACACGTGGAGAA
CTCAGAGGGTGCCTCAGGGGAAAGAAAACTCACGAAGCAGAGGCTGGGCG
TGGTGGCTCTCGCCTGTAATCCCAGCACTTTGGGAGGCCAAGGCAGGCGG
ATCACCTGAGGTCAGGAGTTCGAGACCAGCCTGGCTAACATGGCAAAACC
CCATCTCTACTAAAAATACAAAAATTAGCCGGACGTGGTGGTGCCTGCCT
GTAATCCAGCTACTCAGGAGGCTGAGGCAGGATAATTTTGCTTAAACCCG
GGAGGCGGAGGTTGCAGTGAGCCGAGATCACACCACTGCACTCCAACCTG
GGAAACGCAGTGAGACTGTGCCTCAAAAAAAAGAAAGGAAGAAAAAAGAA
AACTCAGGAAACAGATCTTGGGGGACACTCCAGGGAACCCAAAACTCAAA
GGCGGAGAGCTCAGTGGGCACCACCAAGGCGAGATGAAGCCCCAGCAGGC
ACCTTCAGAAGACCCACGTAGACTGGAGACCCTGCCACGGACAATACTAA
GGACAAAAACCCAGAGACTTGGGCTCTGTGGGCCCCCAAACATGGGGTAA
AGTTGATTTGCCTGATATTCAGGAAGAAGGGGTGAGGGGTGGGTATTTAT
GCTTTTGATTCAGAAGAAAGTGGGGCTTGGGATTCCAGGGACTTGGCTGG
GGGTGGGAAACTTCATCCACTTCCCTACTCTCATCATGAGTACGGACAGG
GTGGGCGGGAGACTGATCATCGGGACTCATCATGAAGAGCCCAGCCCCAC
CGCACATACTCAGATCCCACCCACAGACTGGTGGCGACACCTGAGCCTGG
TCACAAAGAGTTACACTCAGATACATGAGCACGGCAGCGTGCTCATAACT
GTTTAACAACCAGCTGTCCTGGGAGGGGGACAGCTTTGTAATGTTTGCCA
ATTTCCATGGTGTAAATGCTACCACCATGGCTGATTTCATCACTGCCAAG
CATAGACATCCCTAATAGGACACCACGGATCTGTCCCCGGCATCCGGCCC
AGGGCCTGGCACAAAGCATGCTCTAGGGAAATGCTTGCTGATTGAAAGGA
AGGAAGAATGACTCTACAGTCACACCTATGGCATCCCACAAAATCTGTCA
CATGGCTGCATAATCTCAGCCACTCTTTCACAACTATAGACTCATACACG
CGAAGTGCCAGATTCATGCACAACCACACAATCACATGGAAGTCACAGAC
GGCATCACAGACAGTCACAGCACTGTGTGTATGTTATAACACAAGCACAC
AAAACTCAGACAGCATCCCAGCTACACAGCCACTCCCAGAGGTGTCACCG
TCACACTTGGTAATTAATACTGATTACATTAGACACAGACAGACCAAGTT
ATAGTCAGACCTGGTTACACACATACACACACACAATATCACCATGACAA
ATACACATTACACACACACAACATCACAATGACAAACACACATTACACAC
ACAACATCACGATGACAAACACACATTACACACACAACATCACGATGACA
AACACACATTACACACACATCACAATGACAAACACAACATTACACACACA
CAACATCACAATGACACACACATCACACACACATCACAATGACAAACACA
CAACATTACACACATATACACACAGCCTGAGGGCCCTCCCCAGCCCAGAC
TAACACATCTTGGGGTGAGGACCAGACCTTGTTCATAACCCTGGGCCTCT
TAACCACTGATCTTTGAAATAAATGGCAAATAGTTGTACCTGGA

As the melanocytes to be used in the experiment, cells which were readily available on the market were used. In the experiment according to an aspect of the present invention, melanocytes (human epidermal melanocytes, neonatal, moderately pigmented donor, HEMn-MP or MC-MP), which exhibited a normal level of pigmentation and were purchased from life technology, and melanocytes (human epidermal melanocytes, neonatal, lightly pigmented donor, HEMn-LP or MC-LP), which exhibited a low level of pigmentation and were purchased from life technology, were used.

As a culture medium for culturing melanocytes, MGM™-4 BulletKit™ (product number: CC-3249) manufactured by LONZA (US) was purchased and used.

### Experimental Example 1

### Measurement of Amount of Melanin Produced

Two kinds of melanocytes that are one exhibiting normal pigmentation and the other exhibiting weak pigmentation were cultured using MGM™-4 BulletKit™ and then treated with the recombinant TNFSF14 protein, respectively.

Specifically, the melanocytes were treated with 25 ng/ml (25 ng per lml of medium) of the recombinant TNFSF14 protein when the confluency of each of the melanocytes cultured was 70%, and then each of the cells were washed with 5 ml of PBS buffer two times in 4 days after the treatment. Thereafter, 1 ml of PBS buffer was added to the cells, and all the cells were scraped and collected by using a scraper. Thereafter, cell pellets were prepared through centrifugation at 13000 rpm for 5 minutes. The color of the cell pellets was then observed, and the degrees of pigment formation were compared with each other.

In addition, the cell pellets were dissolved in 200 μl of 1N NaOH, and the amount of melanin was measured by measuring the absorbance at 450 nm.

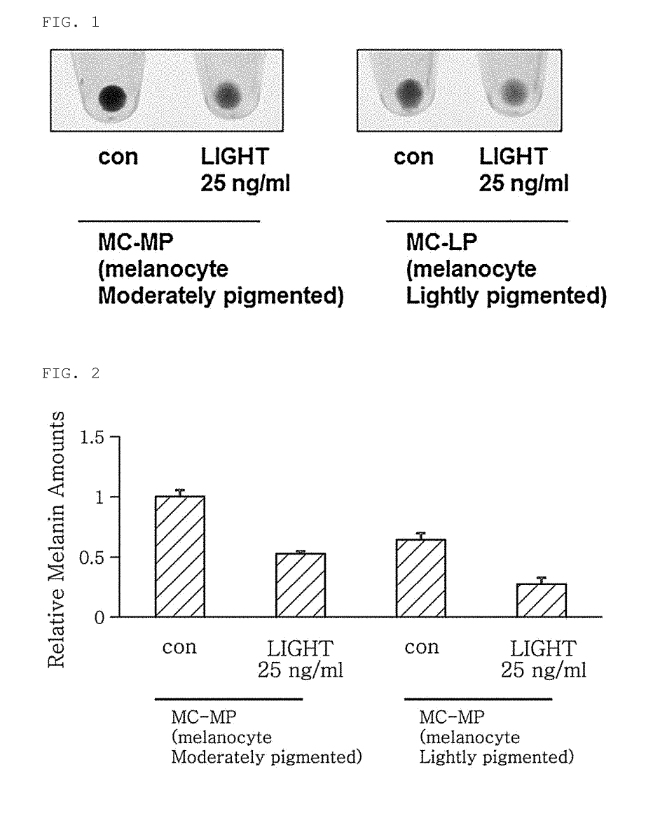

As a result, the results illustrated in FIGS. 1 to 3 were obtained. In FIG. 1, ‘con’ in the upper left part means a control group in which melanocytes (MC-MP) exhibiting normal pigmentation are not treated at all and ‘con’ in the upper right part means a control group in which melanocytes (MC-LP) exhibiting weak pigmentation are not treated at all. ‘LIGHT’ in the upper left part means a group in which melanocytes (MC-MP) exhibiting normal pigmentation are treated with TNFSF14 protein and ‘LIGHT’ in the upper right part means a group in which melanocytes (MC-LP) exhibiting weak pigmentation are treated with TNFSF14 protein. FIG. 2 is a graph which illustrates the relative evaluation on the melanin content in other experimental groups when the melanin content in melanocytes (MC-MP) exhibiting normal pigmentation is regarded as 1.

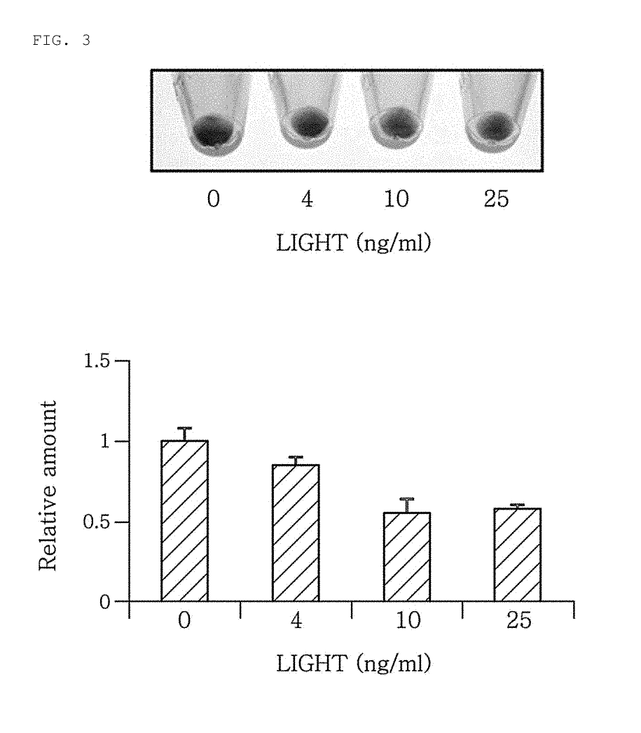

In other words, the amount of melanin produced was decreased by about 50% after the two kinds of melanocytes were all treated with TNFSF14 protein (namely, LIGHT), and it has been thus confirmed that melanin production can be significantly effectively inhibited even by using a small amount of TNFSF14 protein. It has also been confirmed that results similar to these results are obtained when the content of TNFSF14 protein is 25 ng/ml (FIG. 3. It can be seen that similar results will be obtained when the content of TNFSF14 protein is from 10 to 50 ng/ml). It has also been confirmed that melanin production can be effectively inhibited in the same manner when the amount of TNFSF14 gene is increased or the expression thereof is enhanced since TNFSF14 protein is encoded by TNFSF14 gene.

### Formulation Example 1

### Softening lotion (Skin Lotion)

Softening lotion (skin lotion) was prepared using 3.0 wt % of TNFSF14 protein, 1.00 wt % of L-ascorbic acid-2-phosphate magnesium salt, 1.00 wt % of water-soluble collagen (1% aqueous solution), 0.10 wt % of sodium citrate, 0.05 wt % of citric acid, 0.20 wt % of licorice extract, 3.00 wt % of 1,3-butylene glycol, and purified water as the remainder.

### Formulation Example 2

### Cream

A cream formulation was prepared using 3.00 wt % of TNFSF14 protein, 2.00 wt % of polyethylene glycol monostearate, 5.00 wt % of self emulsifying monostearate glycerin, 4.00 wt % of propylene glycol, 6.00 wt % of squalene, 6.00 wt % of tri-2-ethylhexane glyceryl, 1.00 wt % of sphingoglycolipids, 7.00 wt % of 1,3-butylene glycol, 5.00 wt % of beeswax, and purified water as the remainder.

### Formulation Example 3

### Pack

Pack was prepared using 3.00 wt % of TNFSF14 protein, 13.00 wt % of polyvinyl alcohol, 1.00 wt % of L-ascorbic acid-2-phosphate magnesium salt, 1.00 wt % of lauroyl hydroxyproline, 2.00 wt % of water-soluble collagen (1% aqueous solution), 3.00 wt % of 1,3-butylene glycol, 5.00 wt % of ethanol, and purified water as the remainder.

### Formulation Example 4

### Health Food

Health food was prepared by a conventional method according to the composition presented in the following table.

TABLE 2

| Ingredients | Content | |
|---|---|---|
| TNFSF14 protein | 5 | mg |
| Vitamin mixture | | |
| Vitamin A acetate | 70 | μg |
| Vitamin E | 1.0 | mg |
| Vitamin B1 | 0.13 | mg |
| Vitamin B2 | 0.15 | mg |
| Vitamin B6 | 0.5 | mg |
| Vitamin B12 | 0.2 | μg |
| Vitamin C | 10 | mg |
| Biotin | 10 | μg |
| Nicotinic acid amide | 1.7 | mg |
| Folic acid | 50 | μg |
| Calcium pantothenate | 0.5 | mg |
| Mineral mixture | | |
| Ferrous sulfate | 1.75 | mg |
| Zinc oxide | 0.82 | mg |
| Magnesium carbonate | 25.3 | mg |
| Potassium phosphate, monobasic | 15 | mg |
| Calcium phosphate, dibasic | 55 | mg |
| Potassium citrate | 90 | mg |
| Calcium carbonate | 100 | mg |
| Magnesium chloride | 24.8 | mg |

The composition ratios of the vitamins and mineral mixture were obtained by mixing ingredients relatively suitable for health food as an example, but the blending ratio may be arbitrarily changed. The above ingredients may be mixed according to a conventional method of producing health food and then used in the production of a health food composition according to a conventional method.

### Formulation Example 5

### Health Drink

Health drink was prepared by a conventional method according to the composition presented in the following table.

TABLE 3

| Ingredients | Content | |
|---|---|---|
| TNFSF14 protein | 5 | mg |
| Citric acid | 1000 | mg |
| Oligosaccharide | 100 | g |
| Plum concentrate | 2 | g |
| Taurine | 1 | g |
| Purified water | Remainder | |
| Total volume | 900 | ml |

The above ingredients were mixed by adding purified water as the remainder thereto so as to have a total volume of 900 ml as presented in Table 3 according to a conventional method of producing health drink and then stirred and heated at 85° C. for about 1 hour and then the solution was filtered, poured into a sterilized 2 liter vessel, sealed, sterilized, and then stored in a refrigerator, thereby preparing health drink.

Specific portions of the present invention have been described in detail, and it will be apparent to those skilled in the art that this specific description is merely a preferred embodiment and that the scope of the invention is not limited thereby. Accordingly, the actual scope of the present invention will be defined by the appended claims and their equivalents.

What is claimed is:

1. A method for skin whitening, comprising administering a composition comprising one or more of tumor necrosis factor superfamily member 14 (TNFSF14) protein, a protein having 90% or more amino acid sequence homology with the TNFSF14 protein, and a polynucleotide encoding the proteins to a subject in need thereof.

2. The method according to claim 1, wherein the TNFSF14 protein has an amino acid sequence represented by SEQ ID NO: 1.

3. The method according to claim 1, wherein the polynucleotide is mRNA of TNFSF14 gene.

4. The method according to claim 1, wherein the composition inhibits melanin production.

5. The method according to claim 1, wherein the whitening is improving or alleviating one or more selected from the group consisting of melasma, freckle, lentigo, nevi, melanoma, pigmentation by ultraviolet light, pigmentation by drug, pigmentation after inflammation, and pigmentation by dermatitis.

6. The method according to claim 1, wherein a content of the TNFSF14 protein is from 0.00001 to 10 wt % to a total weight of the composition.

7. The method according to claim 1, wherein a dosage of the TNFSF14 protein is from 0.00001 mg/kg/day to 20 mg/kg/day.

8. The method according to claim 1, wherein administration route of the composition is transdermal administration.

9. The method according to claim 1, wherein the polynucleotide is administered by being contained in a vector.

10. The method according to claim 1, wherein the composition for skin whitening is a cosmetic composition.

11. A method for controlling body hair, comprising administering a composition comprising tumor necrosis factor superfamily member 14 (TNFSF14) protein, a protein having 90% or more amino acid sequence homology with the TNFSF14 protein, or a polynucleotide encoding the proteins to a subject in need thereof.

12. The method according to claim 11, wherein the controlling body hair color is one or more selected from the group consisting of graying, whitening, browning, turning red, and yellowing.
